(19) United States
(12) Patent Application Publication
(10) Pub. No.: US 2005/0196433 A1
(43) Pub. Date: Sep. 8, 2005

(54) Pharmaceutical composition and method for the transdermal delivery of magnesium

(76) Inventor: Barbara T. Brierre, Lafayette, LA (US)

Correspondence Address: DOMINGUE & WADDELL, PLC, P.O. Box 3405, LAFAYETTE, LA, 70502, US

(21) Appl. No.: 10/793,374

(22) Filed: Mar. 4, 2004

(51) Int. Cl. A61K 9/70; A61K 33/14

(52) U.S. Cl. 424/449; 424/682

(57) ABSTRACT

The present invention relates to a method and transdermal pharmaceutical composition for preventing magnesium deficiency or imbalances associated with magnesium deficiency including diabetes, hypertension, high cholesterol, cardiac arrhythmias, acute myocardial infarction, arteriosclerosis, atherosclerosis, preeclampsia, dysautonomia, mitral valve prolapse, asthma, constipation, irritable bowel syndrome, migraines, muscle spasms and cramping, premenstrual syndrome, osteoporosis, kidney stones, chronic fatigue syndrome, and fibromyalgia. The transdermal pharmaceutical composition includes a therapeutically effective amount of a pharmaceutically acceptable salt of magnesium and a pharmaceutically acceptable carrier. A therapeutically effective amount of a pharmaceutically acceptable salt of zinc or a vitamin such as vitamin B₆ or vitamin B₁₂, or a combination thereof may also be included in the transdermal pharmaceutical composition. A therapeutically effective amount of progesterone may also be included in the transdermal pharmaceutical composition. The transdermal pharmaceutical composition may be topically administered to prevent magnesium deficiency or imbalances caused by magnesium deficiency.

### FIELD OF THE INVENTION

The present invention relates to a pharmaceutical composition for the transdermal delivery of magnesium and to a method of topically administering the pharmaceutical composition to prevent magnesium deficiency and imbalances associated with magnesium deficiency.

### BACKGROUND OF THE INVENTION

Magnesium is an essential mineral. It is the fourth most abundant cation in the human body and is present in more than 300 enzymatic systems, including adenosine triphosphate (ATP) metabolism, activation of creatine kinase, adenylate cyclase, and sodium potassium-ATPase.

Magnesium functions physiologically in the body to control nerve action, heart activity, neuromuscular transmission, muscular contraction, vascular tone, blood pressure, and peripheral blood flow. Magnesium regulates the entry and release of calcium from cells which is determinative of muscular activity. The importance of magnesium to maintaining health and well-being cannot be overstated.

The recommended daily allowance of magnesium is 320 mg for women and 400 mg for men. Despite the recommended daily allowances, the intake of magnesium for the majority of people is only between 175 mg and 225 mg per day. Most Americans are magnesium-deficient; men obtaining only 80% of the recommended daily allowance and women obtaining only 70%.

Magnesium deficiency has been implicated in the pathogenesis of various clinical imbalances or disorders, including anxiety and panic attacks, asthma, blood clots, bowel disease, cystitis, depression, detoxification, diabetes, fatigue, heart disease, hypertension, hypoglycemia, insomnia, kidney disease, migraines, musculoskeletal conditions, nerve problems, obstetrical and gynecological problems, osteoporosis, Raynaud's syndrome, and tooth decay.

The common nutritional sources of magnesium are green leaf vegetables, legumes, nuts, seeds, and whole grains. However, mineral depletion in soils has resulted in these foods lacking in adequate magnesium content. To meet the daily recommended allowance of magnesium, supplementation is necessary.

Magnesium supplements are commercially available in tablet or capsule form for oral ingestion. Blaine Pharmaceuticals distributes a magnesium supplement (magnesium oxide) in capsule form under the trademark Blaine Mag-Ox 400®. This supplement provides a single dose of 240 mg of elemental magnesium.

Prothera distributes a magnesium supplement in a single-dose capsule containing 150 mg of magnesium amino acid chelate. Prothera also offers combined vitamin and mineral supplements in tablet or capsule form which contain a magnesium supplement. ProThera's OsteoThera dietary supplement is a combination of vitamin D 200 I.U., vitamin K¹ 100 mg, calcium (calcium citrate-malate) 250 mg, magnesium (magnesium amino acid chelate) 100 mg, boron (boron aspartate-citrate) 0.5 mg, and silicon (orthosilicic acid) 5 mg.

Magnesium does not exist alone in nature. It is combined with other substances such as oxide, chloride, or carbonate. When taking an oral magnesium supplement, it may be difficult to discern how much elemental magnesium is available in each tablet or capsule. In a 500 mg capsule of magnesium oxide, only 60% of the magnesium oxide is magnesium; the other 40% is oxide. Therefore, only 300 mg of elemental magnesium (60% of 500 mg) is present. Of this 300 mg, the body may absorb only half or 150 mg. The oral administration of magnesium supplements may also be problematic for other reasons.

Oral delivery of magnesium supplements may result in undesired gastrointestinal side effects such as loose stools or bowel obstruction. Persons with digestive problems due to the lack of hydrochloric acid may have trouble absorbing magnesium. For a percentage of the population, taking oral medications in tablet or capsule form is impossible due to physiological or psychological reasons. The need therefore exists for alternative means for administering magnesium supplements.

### SUMMARY OF THE INVENTION

It is an object of the present invention to provide a delivery mechanism for magnesium which alleviates the disadvantages associated with the oral administration of magnesium supplements.

This object is achieved by the present invention which provides a pharmaceutical composition for the transdermal delivery of magnesium. The transdermal pharmaceutical composition of the invention contains a therapeutically effective amount of a pharmaceutically acceptable salt of magnesium and a pharmaceutically acceptable carrier.

Other minerals and vitamins may be included in the transdermal pharmaceutical composition. In addition to the pharmaceutically acceptable salt of magnesium, the composition may include a therapeutically effective amount of a pharmaceutically acceptable salt of zinc and/or vitamins such as vitamin B₆, vitamin B₁₂, or a combination of vitamin B₆ and vitamin B₁₂. Transdermal pharmaceutical composition may also include a therapeutically effective amount of progesterone.

The pharmaceutically acceptable carrier used in the transdermal pharmaceutical composition of the invention preferably includes a pluronic lecithin organogel. Pluronic lecithin organogel, particularly when comprising Pluronic F127, exhibits optimal skin absorption characteristics.

The transdermal pharmaceutical composition may be topically administered in the appropriate dosage to prevent magnesium deficiency or imbalances associated with or caused by magnesium deficiency, such as diabetes, hypertension, high cholesterol, cardiac arrhythmias, acute myocardial infarction, arteriosclerosis, atherosclerosis, preeclampsia, dysautonomia, mitral valve prolapse, asthma, constipation, irritable bowel syndrome, migraines, muscle spasms and cramping, premenstrual syndrome, osteoporosis, kidney stones, chronic fatigue syndrome, or fibromyalgia.

### DETAILED DESCRIPTION OF PREFERRED EMBODIMENTS OF INVENTION

The present invention is a transdermal pharmaceutical composition for preventing magnesium deficiency and/or imbalances caused by magnesium deficiency. The transdermal pharmaceutical composition may contain a therapeutically effective amount of a pharmaceutically acceptable salt of magnesium and a pharmaceutically acceptable carrier.

Examples of pharmaceutically acceptable salts of magnesium that may be used in the transdermal pharmaceutical composition include magnesium oxide, magnesium carbonate, magnesium chloride, magnesium sulfate, magnesium phosphate, magnesium bicarbonate, magnesium glycinate, magnesium aspartate, magnesium glutamate, magnesium adipate, magnesium citrate, magnesium orotate, magnesium taurate, and magnesium lysinate. The use of magnesium chloride is preferred.

The therapeutically effective amount of the pharmaceutically acceptable salt of magnesium (e.g., magnesium chloride) may be in the range of 5% to 15% of the total weight of the transdermal pharmaceutical composition and more particularly may be about 10% of the total weight of the transdermal pharmaceutical composition.

In addition to the pharmaceutically acceptable salt of magnesium, the transdermal pharmaceutical composition may include other minerals and vitamins. For example, the composition may include a therapeutically effective amount of a pharmaceutically acceptable salt of zinc. Zinc is a co-factor in hormonal metabolism, aids in the immune system, and helps build the collagen matrix of cartilage and bone. It is preferred if the pharmaceutically acceptable salt of zinc is zinc chloride.

The therapeutically effective amount of the pharmaceutically acceptable salt of zinc (e.g., zinc chloride) may be in the range of 0.5% to 5% of the total weight of the transdermal composition and more preferably may be about 2% of the total weight of the transdermal pharmaceutical composition.

Vitamins such as vitamin B₆ or vitamin B₁₂ or a combination of both may also be included in the transdermal pharmaceutical composition of the invention. Vitamin B₆ functions to increase the amount of magnesium that can enter cells and thus provides a synergistic and beneficial effect when combined with magnesium. Vitamin B₆ also facilitates the production of progesterone and reduces inflammatory reactions in connective tissue and collagen repair. Vitamin B₁₂ assists in the proper absorption of other vitamins. Both vitamin B₆ and vitamin B₁₂ promote brain function, transfer food into energy within cells, and neutralize homocysteine which is a toxic by-product of protein metabolism and a risk factor for heart disease.

Stomach absorption of vitamin B₁₂ by oral dosing is problematic as there is minimal absorption of B₁₂ in the stomach due to the body's natural production of intrinsic factors. Thus, B₁₂ is normally delivered by intramuscular injection. The present invention avoids the disadvantages associated with oral delivery and/or intramuscular injection by providing for the transdermal delivery of vitamin B₁₂.

The therapeutically effective amount of vitamin B₆ may preferably be in the range of 2% to 8% of the total weight of the transdermal pharmaceutical composition. More preferably, the therapeutically effective amount of vitamin B₆ may be about 5% of the total weight of the transdermal pharmaceutical composition.

The therapeutically effective amount of vitamin B₁₂ may preferably be in the range of 0.0025% to 0.005% of the total weight of the transdermal pharmaceutical composition. More preferably, the therapeutically effective amount of vitamin B₁₂ may be about 0.005% of the total weight of the transdermal pharmaceutical composition.

The transdermal pharmaceutical composition of the present invention may also include progesterone such as progesterone USP Micronized in a therapeutically effective amount. Preferably, the therapeutically effective amount of progesterone is in the range of 1% to 2% of the total weight of the transdermal pharmaceutical composition. More preferably, the therapeutically effective amount of progesterone may be 2% of the total weight of the transdermal pharmaceutical composition.

As stated above, the transdermal pharmaceutical composition of the invention includes a pharmaceutically acceptable carrier for the active drug or supplement component. The pharmaceutically acceptable carrier preferably includes a pluronic lecithin organogel. The pluronic lecithin organogel may preferably be a mixture of soy lecithin/isopropyl palmitate syrup or solution and Pluronic F127 gel.

Pluronics (e.g., Pluronic F127 gel) are poloxamers. Poloxamers are co-polymers of polyoxyethylene and polyoxypropylene. Pluronics are commercially available from BASF Corporation.

Pluronics form thermoreversible gels in concentrations ranging from 15% to 50%. This means they are liquids at cool (refrigerator) temperature, but are gels at room or body temperature. This characteristic is useful in pharmaceutical compounding because the Pluronics can be drawn into a syringe for accurate dose measurement when cold. When warmed to body temperature—as when applied to the skin—it thickens into a gel consistency that is odorless, colorless, and non-greasy. The thickening of the gel on the skin is rapid. After thickening, the gel penetrates the skin and leaves only a small amount of residue.

By combining Pluronic F127 gel (preferably Pluronic F127 20% gel) and a soy lecithin/isopropyl palmitate syrup or solution (thus resulting in what is known as a “PLO gel”), skin absorption characteristics are enhanced. To explain how skin absorption occurs, it is necessary to understand the composition of the skin.

The skin is composed of three major components: the epidermis, the dermis, and the underlying subdermal tissue. The epidermis, which provides the strongest protection against drug absorption, is composed of five different layers: stratum corneum, stratum lucidum, stratum granulosum, stratum spinosum, and stratum basale. Of these five layers, the stratum corneum is the most impermeable. It is made of flattened, cornified cells embedded in a lipid intercellular matrix.

PLO gels permeate the skin by two proposed mechanisms. The first mechanism proposes that the PLO gel with the active drug diffuses through the lipid intercellular matrix of the stratum corneum. The second mechanism proposes that the PLO gel provides a slight disorganization of the skin allowing permeation of the gel and the active drug through the stratum corneum. The lecithin component of the PLO gel (which is lipophilic) has the ability to act as an amphoteric surfactant and enables drugs to penetrate through the stratum corneum. When a water-soluble drug is added to a hydrophobic substance with the aid of a surfactant, both the drug and the hydrophobic medium can pass through the epidermis. Bioavailability ranges from 10% to 60%.

The transdermal pharmaceutical composition of the invention may be used in a method to prevent magnesium deficiency and/or imbalances caused by or associated with magnesium deficiency. These imbalances include diabetes, hypertension, high cholesterol, cardiac arrhythmias, acute myocardial infarction, arteriosclerosis, atherosclerosis, preeclampsia, dysautonomia, mitral valve prolapse, asthma, constipation, irritable bowel syndrome, migraines, muscle spasms and cramping, premenstrual syndrome, osteoporosis, kidney stones, chronic fatigue syndrome, and fibromyalgia.

The transdermal pharmaceutical composition of the invention should be applied to clean, hairless areas of the body such as the inside of the forearms, upper chest, and upper thigh. The PLO gel will form a deposit on the skin that provides sustained release of the active drug or supplement, e.g., pharmaceutically acceptable salts of magnesium and/or other minerals (zinc chloride) and/or vitamins (vitamin B₆ and/or vitamin B₁₂).

The amount of magnesium supplement (e.g. magnesium chloride) contained in the transdermal pharmaceutical composition required to prevent magnesium deficiency or imbalances caused by magnesium deficiency may vary depending on the specific magnesium supplement used, a person's level of magnesium deficiency, the amount of magnesium supplied by the person's diet, the specific imbalance being treated, the person's life-style (e.g., sedentary versus athletic), and the person's stress level. In general, the amount of magnesium sufficient to meet the recommended daily allowance of magnesium or to prevent magnesium deficiency or imbalances caused by magnesium deficiency is preferably in the range of 300 to 1000 mg/day and more preferably is about 600 mg/day. This includes magnesium obtained from dietary sources as well as through supplementation.

The amount of zinc supplement (e.g., zinc chloride) contained in the transdermal pharmaceutical composition that is necessary to have a therapeutic effect may vary depending on the specific supplement used and other factors related to a person's medical history and lifestyle. In general, the amount required to have a therapeutic effect and/or meet the recommended daily allowance of zinc is preferably in the range of 10 to 30 mg/day and more preferably is about 15 mg/day.

The amount of vitamins (vitamin B₆ and/or vitamin B₁₂) contained in the transdermal pharmaceutical composition may vary depending on the specific vitamin used and other factors related to a person's medical history and lifestyle. In general, the amount necessary to have a therapeutic effect and/or meet the recommended daily allowance of vitamin B₆ is preferably in the range of 50 to 150 mg/day and more preferably is about 100 mg/day. In general, the amount necessary to have a therapeutic effect and/or meet the recommended daily allowance of vitamin B₁₂ is preferably in the range of 25 to 50 mcg/day and more preferably is about 50 mcg/day.

The amount of progesterone contained in the transdermal pharmaceutical composition may vary depending on the specific progesterone used and other factors related to a person's medical history and lifestyle. In general, the amount necessary to have a therapeutic effect is preferably in the range of 20 mg to 80 mg/day and more preferably about 40 mg/day. It has been found that when a therapeutically effective amount of progesterone is included in the transdermal pharmaceutical composition of the present invention the composition exhibits an enhanced ability to prevent imbalances associated with premenstrual syndrome as for example by preventing pain and cramping associated with premenstrual syndrome or menstruation.

The transdermal pharmaceutical composition of the invention may be self-administered. For self-administration, the transdermal pharmaceutical composition may be placed in a dispenser (e.g., syringe) that can be manipulated to provide the suitable dosage. The dosage is preferably 1 ml once per day and more preferably is 0.5 ml twice per day.

The transdermal pharmaceutical composition may also be provided in pre-measured dosages. For example, a pre-measured dose (e.g., 1 ml or 0.5 ml) of the transdermal pharmaceutical composition may be packaged in a blister pack which can be opened, extruded from the blister pack, and placed at the administration site where it is rubbed into the skin.

In a 1 ml dose, the transdermal pharmaceutical composition preferably contains: 100 mg of Mg, 20 mg of Zn, 50 mg of B₆, and 50 mcg of B₁₂. If progesterone is also included, it is preferred if the 1 ml dose includes 20 mg of progesterone.

Provided below are formulation examples which describe the preparation of the transdermal pharmaceutical composition of the invention and therapeutic examples which describe results obtained or expected from transdermal administration to human patients.

### FORMULATIONS

### EXAMPLE 1

### Pluronic 20% Gel

| Chemicals | Quantity | |
|---|---|---|
| Pluronic F127 NF (Poloxamer 407) | 24 | gm |
| Each ml contains 0.2 gm or 20% | | |
| Potassium Sorbate NF | 0.36 | gm |
| Each ml contains 0.003 gm or 0.3% | | |
| Purified Water, USP Liquid | 120 | ml |
| Each ml contains 1 ml or 100% | | |

A vessel is measured to a volume of 100 ml and marked with tape or a permanent marker. This ensures that the 100 ml volume is accurate as the preprinted volume identifiers on the vessel may not be correct.

In the vessel mix together 0.36 gm of potassium sorbate and 24 gm of Pluronic F127. Pluronic F127 is the trade name of poloxamer 407 which is commercially available from BASF.

Add 100 ml of cold (refrigerated) water USP to the mixture. When all granules are thoroughly wet, refrigerate the mixture until the mixture transforms into a solution (at room temperature the mixture will solidify). This may take about 12 to 24 hours.

### EXAMPLE 2

### Lecithin/Isopropyl Palmitate Solution

| Chemicals | Quantity | |
|---|---|---|
| Lecithin soya granular | 54.54 | gm |
| Each ml contains 0.455 gm or 45.5% | | |
| Isopropyl palmitate NF | 63.81 | ml |
| Each ml contains 0.532 ml or 53.2% | | |
| Sorbic Acid NF Powder | 0.36 | gm |
| Each ml contains 0.003 gm or 0.3% | | |

Place 63.81 ml of isopropyl palmitate in a vessel. Disperse 54.54 gm of lecithin soya granular and 0.36 gm of sorbic acid NF powder into the isopropyl palmitate. Allow mixture to stand overnight and form a liquid syrup. Alternatively, isopropyl myristate may be used in place of isopropyl palmitate.

### EXAMPLE 3

### Magnesium CL/Zinc CL 10%/2% PLO Gel

| Chemicals | Quantity | |
|---|---|---|
| Magnesium Chloride USP | 6 | gm |
| Each ml contains 0.1 gm or 10% | | |
| Zinc Chloride USP | 1.2 | gm |
| Each ml contains 0.02 ml or 2% | | |
| Preserved Water Liquid | 3 | ml |
| Each ml contains 0.05 ml or 5% | | |
| Lecithin/Isopropyl Palmitate Solution | 13.2 | ml |
| Each ml contains 0.22 ml or 22% | | |
| Pluronic F127 20% Gel | 60 | ml |
| Each ml contains 1 ml or 100% | | |

Place 3 ml of preserved water liquid in a vessel. Dissolve 6 gm of magnesium chloride and 1.2 gm of zinc chloride in the water.

Add 13.2 ml of lecithin/isopropyl palmitate solution to the mixture and mix well. Qs to 60 ml with Pluronic F127 20% gel.

### EXAMPLE 4

### Magnesium CL/Zinc CL/B₆ PLO Gel

| Chemicals | Quantity | |
|---|---|---|
| Magnesium Chloride USP | 6 | gm |
| Each 273474 contains 0.1 gm or 10% | | |
| Zinc Chloride USP | 1.2 | gm |
| Each 273474 contains 0.02 ml or 2% | | |
| Pyridoxine Hydrochloride USP (vitamin B₆) | 3 | gm |
| Each 273474 contains 0.05 or 5% | | |
| Ethoxy Diglycol Reagent | 3 | ml |
| Each 273474 contains 0.05 or 5% | | |
| Preserved Water Liquid | 3 | ml |
| Each 273474 contains 0.05 ml or 5% | | |
| Lecithin/Isopropyl Palmitate Solution | 13.2 | ml |
| Each 273474 contains 0.22 ml or 22% | | |
| Pluronic F127 20% Gel | 60 | ml |
| Each 273474 contains 1 ml or 100% | | |

Place 3 ml of preserved water liquid in a vessel. Dissolve 6 gm of magnesium chloride and 1.2 gm of zinc chloride in the water.

In separate vessel, dissolve 3 gm pyridoxine hydrochloride USP in 3 ml of ethoxy diglycol reagent. Add this mixture to the mixture of water, magnesium chloride and zinc chloride.

Add 13.2 ml of lecithin/isopropyl palmitate solution to the mixture and mix well. Qs to 60 ml with Pluronic F127 20% gel.

### EXAMPLE 5

### Magnesium CL/Zinc CL/B₆/B₁₂ PLO Gel

| Chemicals | Quantity | |
|---|---|---|
| Magnesium Chloride USP | 6 | gm |
| Each 273474 contains 0.1 gm or 10% | | |
| Zinc Chloride USP | 1.2 | gm |
| Each 273474 contains 0.02 ml or 2% | | |
| Pyridoxine Hydrochloride USP (vitamin B₆) | 3 | gm |
| Each 273474 contains 0.05 or 5% | | |
| Cyanocobalamin (vitamin B₁₂) | 0.003 | gm |
| Each 273474 contains 50 mcg or 0.005% | | |
| Ethoxy Diglycol Reagent | 3 | ml |
| Each 273474 contains 0.05 or 5% | | |
| Preserved Water Liquid | 3 | ml |
| Each 273474 contains 0.05 ml or 5% | | |
| Lecithin/Isopropyl Palmitate Solution | 13.2 | ml |
| Each 273474 contains 0.22 ml or 22% | | |
| Pluronic F127 20% Gel | 60 | ml |
| Each 273474 contains 1 ml or 100% | | |

Place 3 ml of preserved water liquid in a vessel. Dissolve 6 gm of magnesium chloride, 1.2 gm of zinc chloride, and 0.003 gm of cyanocobalamin (vitamin B₁₂) in the 3 ml of water.

In separate vessel, dissolve 3 gm pyridoxine hydrochloride USP in 3 ml of ethoxy diglycol reagent. Add this mixture to the mixture of water, magnesium chloride, zinc chloride, and cyanocobalamin.

Add 13.2 ml of lecithin/isopropyl palmitate solution to the mixture and mix well. Qs to 60 ml with Pluronic F127 20% gel.

### EXAMPLE 6

### Progesterone/Magnesium CL/Zinc CL/B₆/B₁₂ PLO Gel

| Chemicals | Quantity | |
|---|---|---|
| Progesterone USP Micronized | 1.2 | gm |
| Magnesium Chloride USP | 6 | gm |
| Zinc Chloride USP | 1.2 | gm |
| Pyridoxine Hydrochloride USP (vitamin B₆) | 3 | gm |
| Cyanocobalamin (vitamin B₁₂) | 0.003 | gm |
| Preserved Water Liquid | 3 | ml |
| Ethoxy Diglycol Reagent | 4 | ml |
| Lecithin/Isopropyl Palmitate Solution | 13.2 | ml |
| Pluronic F127 20% Gel | 60 | ml |

Place 3 ml of preserved water liquid in a vessel. Dissolve 6 gm of magnesium chloride, 1.2 gm of zinc chloride, and 0.003 gm of cyanocobalamin (vitamin B₁₂) in 3 ml in the water.

In separate vessel, dissolve 3 gm pyridoxine hydrochloride USP and 1.2 gm of Progesterone USP Micronized in 4 ml of ethoxy diglycol reagent. Add this mixture to the mixture of water, magnesium chloride, zinc chloride, and cyanocobalamin.

Add 13.2 ml of lecithin/isopropyl palmitate solution to the mixture and mix well. Qs to 60 ml with Pluronic F127 20% gel.

### THERAPEUTIC EXAMPLES

### MG/ZN/B₆/B12

### EXAMPLE 7

74 year old white man with a progressive neurologic dementia—on multiple medications including antidepressants (Remeron) and an antipyschotic (Seroquel).

Main complaint is constipation unrelieved by Senokot, Metamucil, and increased liquids. He was started on the cream 1 ml b.i.d.—and subsequently (within 2 days) started experiencing daily normal bowel movements with an additional benefit of increased energy and increased appetite.

### EXAMPLE 8

13 year old white female started having menses 1 year ago with irregular periods and increasing bouts of premenstrual cramps and tension. She elected not to be placed on oral contraceptives, and instead tried the magnesium cream on a daily basis.

Her PMS resolved within 2 months as her periods regulated. She also had increased energy and resolve of her PMS constipation.

### EXAMPLE 9

47 year old white female with lifelong history of mitral valve prolapse with resulting dysautonomia (hyperadrenergic autonomic nervous system). She has experienced insomnia, anxiety with panic attacks, shortness of breath at rest, palpitations, and irritable bowel syndrome all associated with her dysautonomia. She had previously tried Betablockers, and SSRI's—refused Xanax. She started magnesium cream with resulting resolution of her irritable bowel syndrome, shortness of breath and palpitations. As a result—she had decreased frequency of anxiety with associated panic.

### EXAMPLE 10

52 year old white female with menopausal symptoms—night sweats, hot flashes—but mostly bothered by periodic migraines resulting in missed (sick) days at work—not relieved by analgesics. She started on magnesium cream with resulting decrease in frequency and intensity of hormonally induced migraines.

### EXAMPLE 11

40 year old white male with 3-year h/o chronic fatigue syndrome, unsure etiology, but most likely secondary to lifelong dysautonomia with low blood pressure, headaches, fatigue, insomnia. Started on magnesium cream with subsequent relief of headaches and some improvement of fatigue.

### EXAMPLE 12

13 year old white male with mitral valve prolapse and early symptoms of dysautonomia—including anxiety in social situations, fatigue, difficulty falling asleep, low blood pressure, and stress intolerance. After starting magnesium cream, he reported—more energy, better concentration at school, less anxiety and chocolate cravings, a much easier time awakening in the morning—and the additional benefit of easier bowel movements. Reported really liking the cream—easy to use—sees the benefits himself.

### THERAPEUTIC EXAMPLES

### Progesterone/MG/ZN/B₆/B₁₂

### EXAMPLE 13

16 year old white female who started menses at age 12. She now complains of bloating, mood swings, headaches, chocolate cravings, and abdominal cramping all associated with her period usually starting the week before starting menses. Her doctor recommended birth control pills, but she decided to try the PMS cream. After using the cream daily for 2 months, she was successfully able to decrease the severity, and for the most part, prevent the symptoms of PMS.

### EXAMPLE 14

44 year old white female with a 2 year history of worsening PMS symptoms, including insomnia, anxiety, irritability, bloating, cravings, lower back pain, cramping, and swelling of digits. These have all become increasingly more severe and lasting up to 2 weeks prior to starting her period. Her doctor recommended Xanax and Zoloft for symptom control. She instead started on the PMS cream and with 3 months of daily use, noticed a significant improvement in and prevention of her PMS.

While preferred embodiments of the present invention have been described, it is to be understood that the embodiments described are illustrative only and that the scope of the invention is to be defined solely by the appended claims when accorded a full range of equivalence, many variations and modifications naturally occurring to those skilled in the art from a perusal hereof.

1. A transdermal pharmaceutical composition for preventing magnesium deficiency or imbalances caused by magnesium deficiency comprising:
a therapeutically effective amount of a pharmaceutically acceptable salt of magnesium, and
a pharmaceutically acceptable carrier.

2. The transdermal pharmaceutical composition according to claim 1, wherein said pharmaceutically acceptable salt of magnesium is magnesium chloride.

3. The transdermal pharmaceutical composition according to claim 2, wherein said therapeutically effective amount of magnesium chloride is in the range of 5% to 15% of a total weight of said composition.

4. The transdermal pharmaceutical composition according to claim 3, wherein said therapeutically effective amount of magnesium chloride is about 10% of the total weight of said composition.

5. The transdermal pharmaceutical composition according to claim 1, further comprising a therapeutically effective amount of a pharmaceutically acceptable salt of zinc.

6. The transdermal pharmaceutical composition according to claim 5, wherein said pharmaceutically acceptable salt of zinc is zinc chloride.

7. The transdermal pharmaceutical composition according to claim 1, further comprising a therapeutically effective amount of a vitamin selected from the group consisting of vitamin B₆ and vitamin B₁₂.

8. The transdermal pharmaceutical composition according to claim 7, wherein said therapeutically effective amount of vitamin B₆ is in the range of 2% to 8% of a total weight of said composition.

9. The transdermal pharmaceutical composition according to claim 8, wherein said therapeutically effective amount of vitamin B₆ is about 5% of the total weight of said composition.

10. The transdermal pharmaceutical composition according to claim 7, wherein said therapeutically effective amount of vitamin B₁₂ is in the range of 0.0025% to 0.005% of a total weight of said composition.

11. The transdermal pharmaceutical composition according to claim 10, wherein said therapeutically effective amount of vitamin B₁₂ is about 0.005% of the total weight of said composition.

12. The transdermal pharmaceutical composition according to claim 1, wherein said pharmaceutically acceptable carrier comprises a pluronic lecithin organogel.

13. The transdermal pharmaceutical composition according to claim 12, wherein said pluronic lecithin organogel comprises a mixture of a soy lecithin/isopropyl palmitate and a pluronic F127 gel.

14. The transdermal pharmaceutical composition according to claim 1, wherein said imbalances caused by magnesium deficiency are selected from the group consisting of diabetes, hypertension, high cholesterol, cardiac arrhythmias, acute myocardial infarction, arteriosclerosis, atherosclerosis, preeclampsia, dysautonomia, mitral valve prolapse, asthma, constipation, irritable bowel syndrome, migraines, muscle spasms and cramping, premenstrual syndrome, osteoporosis, kidney stones, chronic fatigue syndrome, and fibromyalgia.

15. A transdermal pharmaceutical composition for preventing magnesium deficiency or imbalances caused by magnesium deficiency comprising:
a therapeutically effective amount of a pharmaceutically acceptable salt of magnesium,
a therapeutically effective amount of a pharmaceutically acceptable salt of zinc,
a therapeutically effective amount of vitamin B₆, or vitamin B₁₂, or a combination of vitamin B₆ and vitamin B₁₂, and
a pharmaceutically acceptable carrier comprising a pluronic lecithin/isopropyl organogel.

16. The transdermal pharmaceutical composition according to claim 15, further comprising a therapeutically effective amount of a progesterone.

17. The transdermal pharmaceutical composition according to claim 16, wherein said therapeutically effective amount of progesterone is in the range of 1% to 2% of the total weight of said composition.

18. The trandermal pharmaceutical composition according to claim 17, wherein said therapeutically effective amount of progesterone is about 2% of the total weight of said composition.

19. A method of preventing magnesium deficiency or imbalances caused by magnesium deficiency comprising the steps of topically administering a transdermal pharmaceutical composition comprising:
a therapeutically effective amount of a pharmaceutically acceptable salt of magnesium, and
a pharmaceutically acceptable carrier.

20. The method according to claim 19, wherein said pharmaceutically acceptable salt of magnesium is magnesium chloride.

21. The method according to claim 20, wherein said therapeutically effective amount of magnesium chloride is in the range of 5% to 15% of a total weight of said composition.

22. The method according to claim 21, wherein said therapeutically effective amount of magnesium chloride is about 10% of the total weight of said composition.

23. The method according to claim 19, wherein said transdermal pharmaceutical composition further comprises a pharmaceutical acceptable salt of zinc.

24. The method according to claim 23, wherein said pharmaceutical acceptable salt of zinc is zinc chloride.

25. The method according to claim 19, wherein said transdermal pharmaceutical composition further comprises a therapeutically effective amount of a vitamin selected from the group consisting of vitamin B₆ and vitamin B₁₂.

26. The method according to claim 25, wherein said therapeutically effective amount of vitamin B₆ is in the range of 2% to 8% of a total weight of said composition.

27. The method according to claim 26, wherein said therapeutically effective amount of vitamin B₆ is about 5% of the total weight of said composition.

28. The method according to claim 25, wherein said therapeutically effective amount of vitamin B₁₂ is in the range of 0.0025% to 0.005% of a total weight of said composition.

29. The method according to claim 28, wherein said therapeutically effective amount of vitamin B₁₂ is about 0.005% of the total weight of said composition.

30. The method according to claim 19, wherein said transdermal pharmaceutical composition further comprises a therapeutically effective amount of a progesterone.

31. The method according to claim 30, wherein said therapeutically effective amount of progesterone is in the range of 1% to 2% of a total weight of said composition.

32. The method according to claim 31, wherein said therapeutically effective amount of progesterone is about 2% of the total weight of said composition.

33. The method according to claim 19, wherein said pharmaceutically acceptable carrier comprises a pluronic lecithin organogel.

34. The method according to claim 33, wherein said pluronic lecithin organogel comprises a mixture of a soy lecithin/isopropyl palmitate and a pluronic F127 gel.

35. The method according to claim 19, wherein said imbalances are selected from the group consisting of diabetes, hypertension, high cholesterol, cardiac arrhythmias, acute myocardial infarction, arteriosclerosis, atherosclerosis, preeclampsia, dysautonomia, mitral valve prolapse, asthma, constipation, irritable bowel syndrome, migraines, muscle spasms and cramping, premenstrual syndrome, osteoporosis, kidney stones, chronic fatigue syndrome, and fibromyalgia.
